(19) United States
(12) Patent Application Publication
(10) Pub. No.: US 2025/0025642 A1
(43) Pub. Date: Jan. 23, 2025

(54) NEBULIZER AND CHEMICAL ATOMIZATION METHOD

(71) Applicant: OMRON HEALTHCARE Co., Ltd., Kyoto (JP)

(72) Inventors: Masakazu DOI, Kyoto (JP); Atsushi KAWANO, Kyoto (JP); Yoichi SASAI, Kyoto (JP); Katsunori KONDO, Kyoto (JP)

(21) Appl. No.: 18/908,340

(22) Filed: Oct. 7, 2024

Related U.S. Application Data
(63) Continuation of application No. PCT/JP2023/003175, filed on Feb. 1, 2023.

(30) Foreign Application Priority Data
Apr. 12, 2022 (JP) 2022-065507

(51) Int. Cl. A61M 11/02; F04B 35/04

(52) U.S. Cl. CPC A61M 11/02; F04B 35/04; A61M 2205/103

(57) ABSTRACT

A compressor includes a shading motor as a power source, and is provided with a discharge port that discharges compressed air generated by driving the shading motor. The shading motor includes a stator that is made of a soft magnetic material and has an apparent volume of 78000 mm³ or less. A load reduction mechanism reduces a load applied to the shading motor by suppressing a pressure increase in the flow path over a period from a first time point at which the shading motor starts driving to a second time point at which the rotation speed of a rotor reaches a predetermined rotation speed.

### CROSS-REFERENCE TO RELATED APPLICATIONS

This application is the U.S. national stage application filed pursuant to 35 U.S.C. 365 (c) and 120 as a continuation of International Patent Application No. PCT/JP2023/003175, filed Feb. 1, 2023, which application claims priority to Japanese Patent Application No. 2022-065507, filed Apr. 12, 2022, which applications are incorporated herein by reference in their entireties.

### TECHNICAL FIELD

The present disclosure relates to a nebulizer and a chemical atomization method.

### BACKGROUND ART

Prior art documents disclosing a nebulizer include JP 2006-87446 A (Patent Document 1). The nebulizer described in Patent Document 1 includes an air flow passage and a chemical solution tank. The air flow passage extends from an air inlet to a spray port. A chemical solution in the chemical solution tank is atomized by the air compressed by a compressor. The air introduced from the air inlet is mixed with the chemical solution atomized in the chemical solution tank, and the mixed air is discharged from the spray port.

### CITATION LIST

Patent Document 1: JP 2006-87446 A

### SUMMARY OF INVENTION

### Technical Problem

A shading motor (a shaded pole motor) may be used as a power source of a compressor in a nebulizer. When the compressor is driven, the load applied to the shading motor increases as the pressure in a space on a discharge side of the compressor increases. Thus, when a small-sized shading motor is used, the torque of the shading motor becomes insufficient, and it becomes difficult to rotate a rotor in excess of a predetermined rotation speed. As a result, there is a possibility that the chemical solution cannot be sufficiently atomized.

The present disclosure has been made to solve the above-described problem, and an object thereof is to provide a nebulizer and a chemical atomization method capable of sufficiently atomizing a chemical solution even when a small-sized shading motor is used.

### Solution to Problem

A nebulizer according to the present disclosure includes a compressor, a nozzle,

an atomization unit, a flow path, and a load reduction mechanism. The compressor includes a shading motor as a power source, and is provided with a discharge port for discharging compressed air generated by driving the shading motor. The nozzle injects the compressed air generated by the compressor. The atomization unit includes a baffle arranged so as to face the nozzle, and generates an aerosol by adding a chemical solution to the compressed air injected from the nozzle and spraying the compressed air added with the chemical solution onto the baffle. The flow path connects the discharge port and the nozzle. The shading motor includes a rotor and a stator that is made of a soft magnetic material and has an apparent volume of 78000 mm³ or less. The load reduction mechanism reduces a load applied to the shading motor by suppressing a pressure increase in the flow path over a period from a first time point at which the shading motor starts driving to a second time point at which the rotation speed of the rotor reaches a predetermined rotation speed.

According to the above configuration, the load applied to the shading motor can be reduced by the load reduction mechanism until the rotor of the shading motor reaches the predetermined rotation speed. Thus, even when a small-sized shading motor is used, the rotor can be rotated in excess of the predetermined rotation speed. Accordingly, even when a small-sized shading motor is used, the chemical solution can be sufficiently atomized.

In one example of the present disclosure, an opening/closing unit is provided on the flow path. The opening/closing unit enables discharge of the compressed air in the flow path to the outside of the flow path in an open state and disables discharge of the compressed air in the flow path to the outside of the flow path in a closed state. The opening/closing unit is maintained in the open state from the first time point to the second time point, and the opening/closing unit is maintained in the closed state after the second time point is exceeded, so that the load reduction mechanism is configured by the opening/closing unit.

According to the above configuration, by providing the opening/closing unit as the load reduction mechanism on the flow path, it is possible to reduce the load applied to the shading motor until the rotor of the shading motor reaches the predetermined rotation speed. Thus, the use of a small-sized shading motor can be enabled with a simple configuration in which the opening/closing unit is provided on the flow path.

In one example of the present disclosure, the opening/closing unit is made of a diaphragm valve.

According to the above-described configuration, since the opening/closing unit as the load reduction mechanism can be configured by the diaphragm valve having a simple configuration, it is possible to contribute to reduction of manufacturing cost and downsizing of the nebulizer.

In one example of the present disclosure, the load reduction mechanism is configured by a buffer tank provided on the flow path.

According to the above configuration, by providing the buffer tank as the load reduction mechanism on the flow path, it is possible to reduce the load applied to the shading motor until the rotor of the shading motor reaches the predetermined rotation speed. Thus, the use of a small-sized shading motor can be enabled with a simple configuration in which the buffer tank is provided on the flow path.

In one example of the present disclosure, the compressor further includes a piston, a cylinder, and a crank. The piston and the cylinder define a pump chamber in which compressed air is generated. The crank is connected to the rotor and the piston so as to convert a rotational motion of the rotor into a reciprocating motion of the piston. The compressor is configured such that an eccentricity of the crank with respect to the rotor increases as the rotation speed of the rotor increases, and thereby a compression ratio in the pump chamber increases, so that the load reduction mechanism is configured by the crank.

According to the above configuration, by providing the compressor with the crank having a variable eccentricity as the load reduction mechanism, it is possible to reduce the load applied to the shading motor until the rotor of the shading motor reaches the predetermined rotation speed. Thus, the use of a small-sized shading motor can be enabled with a simple configuration in which the above-described crank is provided at the compressor.

In a chemical atomization method in a nebulizer according to the present disclosure, an aerosol is generated by injecting compressed air from a nozzle and spraying the compressed air onto a baffle while adding a chemical solution to the compressed air. The nebulizer includes a compressor and a flow path. The compressor includes a shading motor as a power source, and is provided with a discharge port for discharging the compressed air generated by driving the shading motor. The flow path connects the discharge port and the nozzle. The shading motor includes a rotor and a stator that is made of a soft magnetic material and has an apparent volume of 78000 mm³ or less. The chemical atomization method includes starting driving of the shading motor, and reducing a load applied to the shading motor by suppressing a pressure increase in the flow path over a period from a first time point at which the shading motor starts driving to a second time point at which a rotation speed of the rotor reaches a predetermined rotation speed.

According to the above method, the load applied to the shading motor can be reduced until the rotor of the shading motor reaches the predetermined rotation speed. Thus, even when a small-sized shading motor is used, the rotor can be rotated in excess of the predetermined rotation speed. Accordingly, even when a small-sized shading motor is used, the chemical solution can be sufficiently atomized.

### Advantageous Effects of Invention

According to the present disclosure, even when a small-sized shading motor is used, the nebulizer can sufficiently atomize the chemical solution.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a perspective view illustrating a configuration of a nebulizer according to a first embodiment of the present disclosure.

FIG. 2 is a schematic diagram illustrating a configuration of the nebulizer according to the first embodiment of the present disclosure.

FIG. 3 is a cross-sectional view of the nebulizer illustrated in FIG. 2 as viewed from an arrow direction of line III-III.

FIG. 4 is a perspective view illustrating a configuration of a compressor included in the nebulizer according to the first embodiment of the present disclosure.

FIG. 5 is a cross-sectional view of the compressor of FIG. 4 as viewed from an arrow direction of line V-V.

FIG. 6 is a cross-sectional view illustrating a configuration of a load reduction mechanism included in the nebulizer according to the first embodiment of the present disclosure.

FIG. 7 is a graph showing the relationship between torque and rotation speed of a general shading motor.

FIG. 8 is a flowchart showing a chemical atomization method of the nebulizer according to the first embodiment of the present disclosure.

FIG. 9 is a schematic diagram illustrating a configuration of a nebulizer according to a second embodiment of the present disclosure.

FIG. 10 is a schematic diagram illustrating a configuration when a crank in a compressor included in a nebulizer according to a third embodiment of the present disclosure rotates at a low speed.

FIG. 11 is a schematic diagram illustrating a configuration when the crank in the compressor included in the nebulizer according to the third embodiment of the present disclosure rotates at a high speed.

### DESCRIPTION OF EMBODIMENTS

Hereinafter, a nebulizer according to each embodiment of the present disclosure will be described with reference to the drawings. In the description of the following embodiments, the same or equivalent components in the drawings are given the same reference signs, and the descriptions thereof are not repeated.

### First Embodiment

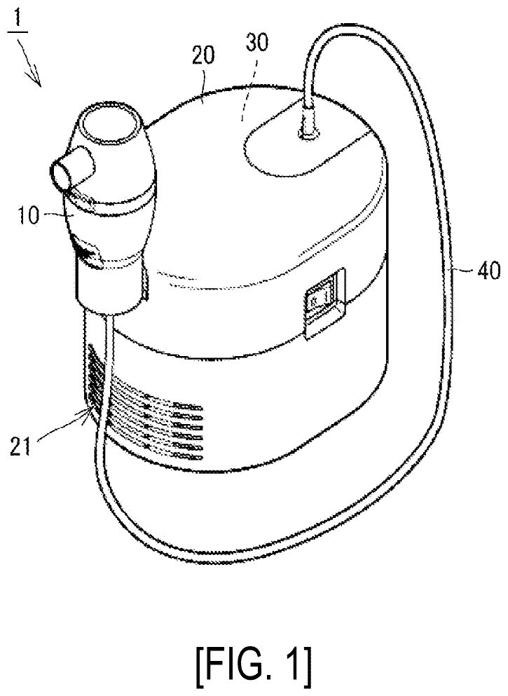

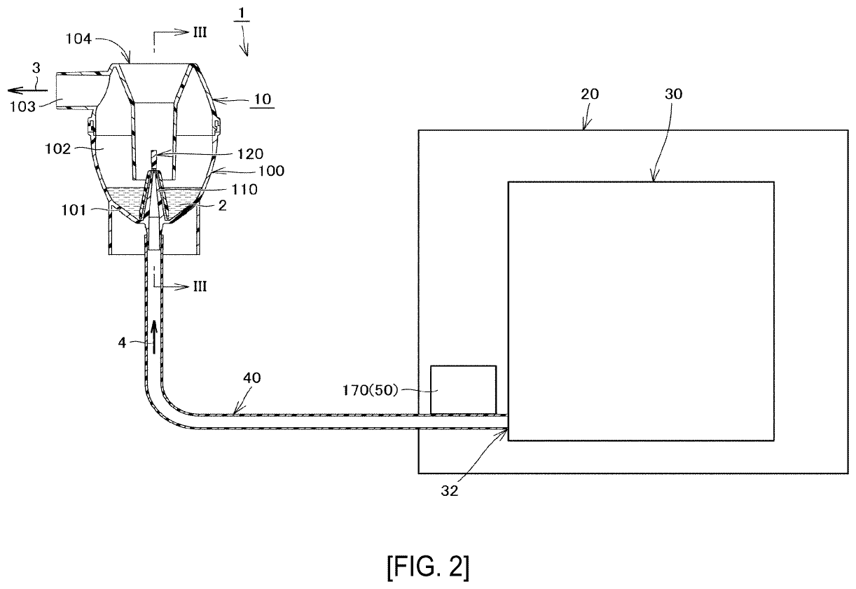

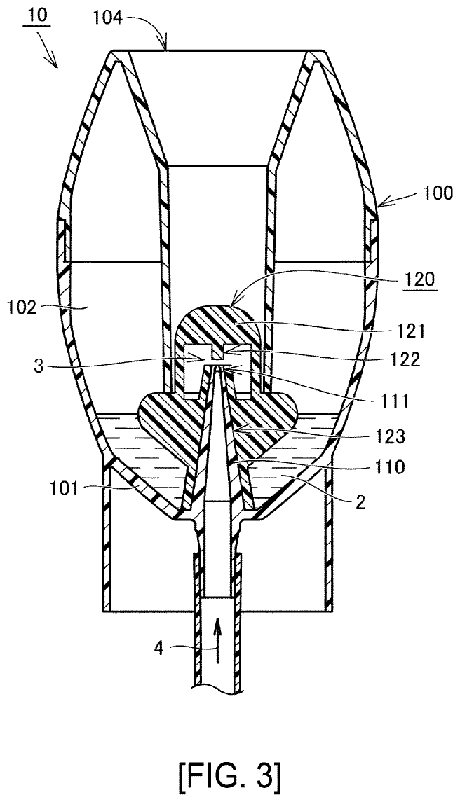

FIG. 1 is a perspective view illustrating a configuration of a nebulizer according to a first embodiment of the present disclosure. FIG. 2 is a schematic diagram illustrating a configuration of the nebulizer according to the first embodiment of the present disclosure. FIG. 3 is a cross-sectional view of the nebulizer illustrated in FIG. 2 as viewed from an arrow direction of line III-III.

As illustrated in FIG. 1 to FIG. 3, a nebulizer 1 according to the first embodiment of the present disclosure includes a nebulizer kit 10, a main body portion 20, a compressor 30, a flow path 40, and a load reduction mechanism 50. The nebulizer 1 is a device used for inhalation therapy in which an atomized chemical solution 2 is directly applied to a nasal cavity, an upper respiratory tract, a bronchus, or the like. The nebulizer kit 10 includes a case body 100, a storage portion 101, an aerosol

discharge port 102, an aerosol transport path 103, a nozzle 110, and an atomization unit 120.

The case body 100 includes a ventilation path 104 opened at an upper end of the case body 100 and is formed in a bottomed tubular shape. The chemical solution 2 is stored in the storage portion 101. After being atomized in the atomization unit 120, the chemical solution 2 is mixed with air introduced from the ventilation path 104 to form an aerosol 3, and the aerosol 3 flows to the aerosol discharge port 102. The aerosol 3 having flowed to the aerosol discharge port 102 is discharged to the outside of the case body 100 through the aerosol transport path 103.

The nozzle 110 injects compressed air 4 generated by the compressor 30 to be described below. As illustrated in FIG. 3, a hole 111 is provided at a tip of the nozzle 110. The inner diameter of the nozzle 110 decreases toward the hole 111.

The atomization unit 120 includes a baffle 121 and a solution suction portion 123. The baffle 121 is disposed so as to face the nozzle 110. A protrusion 122 protruding with respect to the nozzle 110 is provided on the nozzle 110 side of the baffle 121.

The solution suction portion 123 covers the circumference of the nozzle 110 while leaving a gap with the outer peripheral portion of the nozzle 110. By injecting the compressed air from the hole 111 of the nozzle 110, a space in the vicinity of the hole 111 between the inner peripheral portion of the solution suction portion 123 and the outer peripheral portion of the nozzle 110 has a negative pressure.

The atomization unit 120 generates the aerosol 3 by adding the chemical solution 2 to the compressed air 4 injected from the nozzle 110 and spraying the compressed air 4 added with the chemical solution 2 onto the baffle 121. Specifically, the chemical solution 2 is sucked in the space between the inner peripheral portion of the solution suction portion 123 and the outer peripheral portion of the nozzle 110 toward the tip of the nozzle 110, and the chemical solution 2 and the compressed air 4 are sprayed onto the protrusion 122, whereby the chemical solution 2 becomes minute particles, and the air and the minute particles of the chemical solution 2 are mixed to generate the aerosol 3.

As illustrated in FIG. 1 and FIG. 2, the main body portion 20 accommodates the compressor 30, the load reduction mechanism 50, electronic components (not illustrated), and the like. In the main body portion 20, an electrical operation such as power-on of the nebulizer 1 is performed. A ventilation window 21 is provided at a side surface of the main body portion 20.

The nebulizer kit 10 and the compressor 30 are connected by the flow path 40. Specifically, the flow path 40 connects a discharge port 32 of the compressor 30, which will be described below, and the nozzle 110. The flow path 40 in the present embodiment is formed of, for example, a resin tube or a rubber tube.

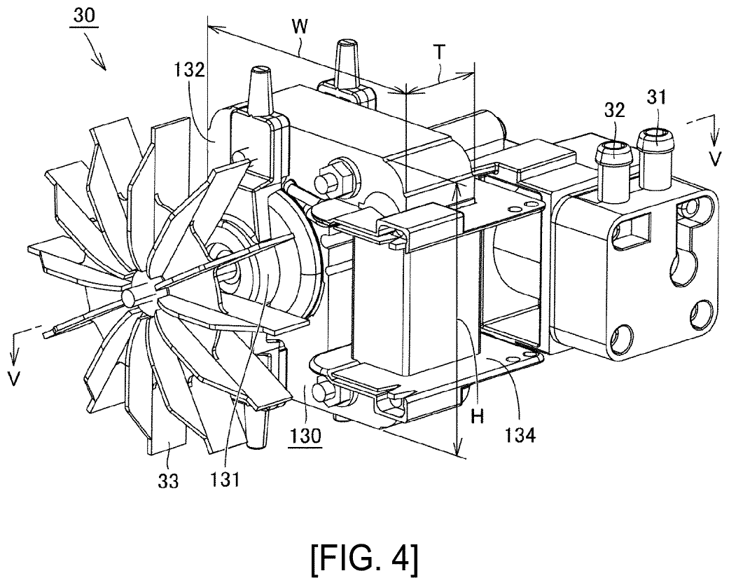

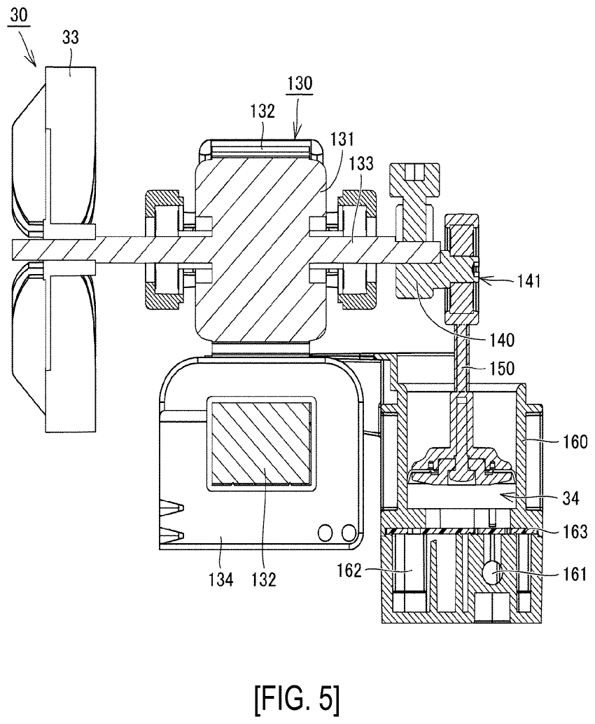

FIG. 4 is a perspective view illustrating a configuration of the compressor included in the nebulizer according to the first embodiment of the present disclosure. FIG. 5 is a cross-sectional view of the compressor of FIG. 4 as viewed from an arrow direction of line V-V.

As illustrated in FIG. 4 and FIG. 5, the compressor 30 includes a suction port 31, the discharge port 32, a fan 33, a shading motor 130 as a power source, a crank 140, a piston 150, and a cylinder 160.

The suction port 31 sucks outside air via the main body portion 20. The discharge port 32 discharges the compressed air 4 generated by driving the shading motor 130. The fan 33 introduces outside air from the ventilation window 21 provided at the main body portion 20 into the main body portion 20 so as to cool the interior of the main body portion 20.

The shading motor 130 includes a rotor 131, a stator 132, a rotor shaft 133, and a support base 134.

The rotor shaft 133 is connected to the rotor 131 at the center of rotation. The rotor shaft 133 is connected, at the respective ends thereof, to the fan 33 and the crank 140. When the rotor 131 and the rotor shaft 133 rotate about the rotor shaft 133 as a rotation center axis, the fan 33 and the crank 140 rotate.

The stator 132 generates a magnetic field by being supplied with electric power from electric wiring (not illustrated). The stator 132 covers the outer peripheral portion of the rotor 131. The stator 132 rotates the rotor 131 by the magnetic field generated at the stator 132.

The stator 132 is made of a soft magnetic material. The stator 132 in the present embodiment is made of, for example, a silicon alloy. The material of the stator 132 is not limited to a silicon alloy as long as the material is a soft magnetic material, and may be pure iron, carbon steel, a nickel alloy, a cobalt alloy, or ceramics (ferrite) containing iron oxide as a main component.

The apparent volume of the stator 132 is 78000 mm³ or less. The apparent volume in the present disclosure is a volume of a minimum rectangular parallelepiped in which the stator 132 can be accommodated, the volume being calculated by multiplying a width, a height, and a thickness of the rectangular parallelepiped. The apparent volume includes a case where a component other than the stator 132 is interposed inside the rectangular parallelepiped.

The minimum rectangular parallelepiped in which the stator 132 of the present

embodiment can be accommodated has a width W of 62.5 mm, a height H of 60.5 mm, and a thickness T of 20.5 mm, for example. Thus, the stator 132 in the present embodiment has an apparent volume of 77516 mm³ (approximately 78000 mm³).

The support base 134 supports the stator 132. The interior of the support base 134 is penetrated by a part of the stator 132.

An eccentric portion 141 of the crank 140 eccentrically rotates about the rotation axis of the rotor shaft 133 when the rotor 131 and the rotor shaft 133 rotate. The crank 140 is connected to the rotor 131 and the piston 150, thereby converting the rotational motion of the rotor 131 into the reciprocating motion of the piston 150. The piston 150 is connected to the eccentric portion 141 of the crank 140. The

cylinder 160 accommodates a part of the piston 150. The piston 150 and the cylinder 160 define a pump chamber 34 in which the compressed air 4 is generated.

The cylinder 160 includes a suction chamber 161, a discharge chamber 162, and a valve 163. The suction chamber 161 is connected to the suction port 31 and introduces outside air into the pump chamber 34. The discharge chamber 162 is connected to the discharge port 32 and discharges the compressed air 4 compressed in the pump chamber 34 to the flow path 40. The valve 163 is disposed between the suction chamber 161 and the pump chamber 34 and between the discharge chamber 162 and the pump chamber 34.

The valve 163 is disposed between the pump chamber 34 and the suction chamber 161 and between the pump chamber 34 and the discharge chamber 162 so as to allow air to flow in one direction. Specifically, in the case where air flows from the suction chamber 161 into the pump chamber 34, a part of the valve 163 opens toward the pump chamber 34 side to take the air into the pump chamber 34. On the other hand, the valve 163 does not open toward the suction chamber 161 side, air does not flow from the pump chamber 34 to the suction chamber 161. Similarly, in the case where the compressed air is discharged from the pump chamber 34 to the discharge chamber 162, a part of the valve 163 opens toward the discharge chamber 162 side to discharge the compressed air to the discharge chamber 162. On the other hand, the valve 163 does not open toward the pump chamber 34 side, the compressed air does not flow from the discharge chamber 162 to the pump chamber 34.

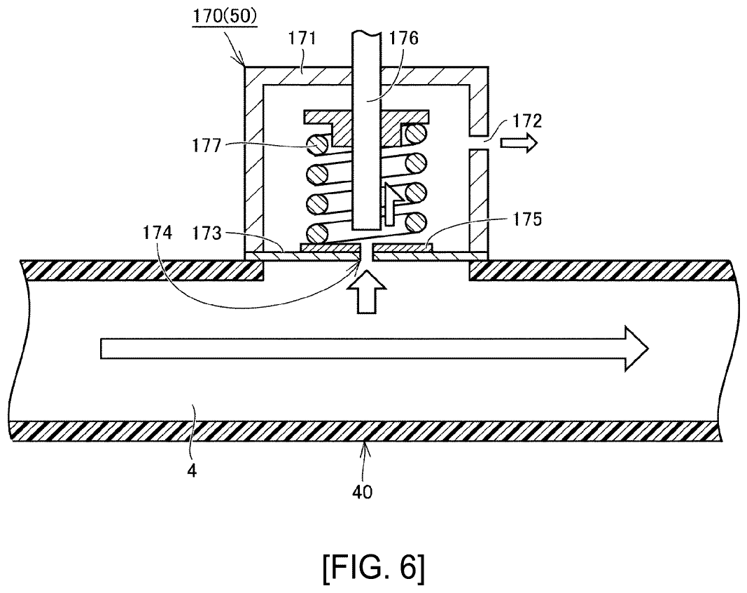

FIG. 6 is a cross-sectional view illustrating a configuration of the load reduction mechanism included in the nebulizer according to the first embodiment of the present disclosure.

As illustrated in FIG. 2 and FIG. 6, the load reduction mechanism 50 causes a phenomenon in which a pressure increase in the flow path 40 is suppressed separately from a phenomenon in which a pressure increase in the flow path 40 is suppressed by discharge of a part of the compressed air 4 generated by the compressor 30 from the nozzle 110 until the shading motor 130 reaches a predetermined rotation speed to be described below. That is, the nozzle 110 does not correspond to the load reduction mechanism in the present disclosure.

The load reduction mechanism 50 in the present embodiment includes an opening/closing unit 170. The opening/closing unit 170 as the load reduction mechanism 50 is provided on the flow path 40. The opening/closing unit 170 in the present embodiment is provided inside the main body portion 20. Note that the load reduction mechanism 50 is not limited to being disposed inside the main body portion 20, and may be disposed immediately below the nebulizer kit 10 in the flow path 40, at an intermediate position in the flow path 40, or the like.

The opening/closing unit 170 is formed of a diaphragm valve. The opening/closing unit 170 includes a case 171, a thin film portion 173, a sheet portion 175, a closing portion 176, and a spring 177.

The case 171 accommodates other components therein. The case 171 is provided with a through hole 172.

The thin film portion 173 is provided with an opening 174 through which the compressed air 4 flows. The thin film portion 173 expands along a direction in which the compressed air 4 flows through the opening 174 as the pressure of the compressed air 4 increases. The sheet portion 175 is provided adjacent to the thin film portion 173.

The opening/closing unit 170 allows the compressed air 4 in the flow path 40 to be discharged to the outside of the flow path 40 in an open state, and prevents the compressed air 4 in the flow path 40 from being discharged to the outside of the flow path 40 in a closed state.

In the opening/closing unit 170 in the present embodiment, a state in which the opening 174 of the thin film portion 173 is not closed by the closing portion 176 corresponds to the open state. When the opening/closing unit 170 is in the open state, the compressed air 4 is discharged to the outside of the flow path 40 via the opening 174 and the through hole 172.

In the opening/closing unit 170, a state in which the opening 174 of the thin

film portion 173 is closed by the closing portion 176 corresponds to the closed state. In the closed state, the opening/closing unit 170 prevents the compressed air 4 from being discharged to the outside of the flow path 40. Specifically, the closing portion 176 comes into contact with the sheet portion 175 by the expansion of the thin film portion 173, thereby closing the opening 174. When the pressure of the compressed air 4 is applied to the thin film portion 173, the thin film portion 173 bulges toward the closing portion 176 side against the spring 177, thereby closing the opening 174. Accordingly, the compressed air 4 is prevented from being discharged to the outside of the flow path 40.

Note that the opening/closing unit 170 is not limited to the diaphragm valve, and may have a configuration of a pressure reduction valve without a diaphragm, or may have a configuration in which the opening is opened and closed by a float that moves up and down in accordance with the flow rate of the compressed air 4. In addition, the opening/closing unit 170 is not limited to having a configuration that is automatically opened and closed by pressure like a diaphragm valve, and may have a configuration in which a lid portion capable of opening and closing the opening is manually opened and closed.

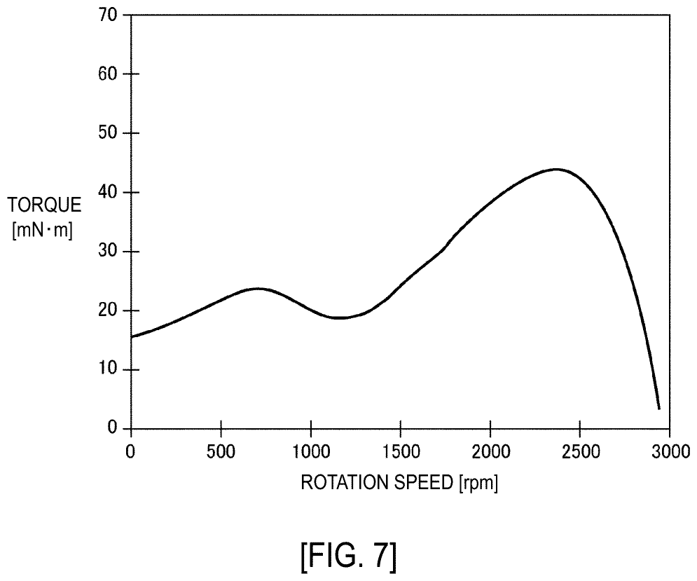

FIG. 7 is a graph showing the relationship between torque and rotation speed of a general shading motor.

As shown in FIG. 7, the shading motor as a single-phase AC motor is, due to its structure, characterized in that a torque, which is the output of the shading motor, is unstable and low at a predetermined rotation speed or less, the torque becomes stable and high when the predetermined rotation speed is exceeded, and then the torque gradually decreases as the rotation speed increases. The predetermined rotation speed of the shading motor in this case varies depending on the configuration of the shading motor and the like, and is about 2300 rpm in the present example. In the shading motor, the torque is unstable in a range of about 15 to 45 mN·m over a period from a first time point at which driving is started to a second time point at which the rotation speed of the rotor reaches the predetermined rotation speed (about 2300 rpm).

On the other hand, when the pressure on the discharge side increases due to the pressure of the compressed air compressed by the driving of the compressor, the compressed air becomes a resistance against the torque of the shading motor in the compressor, and thus the load applied to the shading motor increases as the rotation speed of the shading motor increases.

When this shading motor is downsized with an apparent volume of 77516 mm³ (approximately 78000 mm³) for the purpose of reducing an environmental load or the like as in the case of the shading motor 130 described in the present embodiment, the torque is reduced as compared with the case where the shading motor is not downsized. As a result, since the torque becomes lower than the load applied to the shading motor at a rotation speed equal to or lower than the predetermined rotation speed, it becomes difficult for the downsized shading motor to rotate in excess of the predetermined rotation speed.

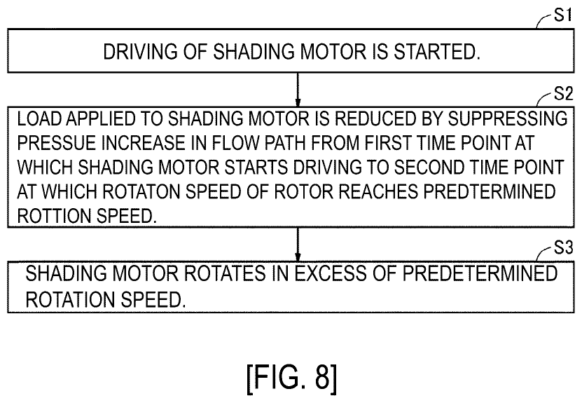

Hereinafter, a chemical atomization method of the nebulizer 1 including the shading motor 130 which has been downsized will be described. FIG. 8 is a flowchart showing a chemical atomization method of the nebulizer according to the first embodiment of the present disclosure.

As the chemical atomization method of the nebulizer 1, first, the shading motor 130 starts driving as illustrated in FIG. 6 and FIG. 8 (step S1).

Next, the load applied to the shading motor 130 is reduced by suppressing a pressure increase in the flow path 40 over a period from the first time point at which the shading motor 130 starts driving to the second time point at which the rotation speed of the rotor 131 reaches the predetermined rotation speed (step S2). Specifically, the opening/closing unit 170 is maintained in the open state over the period from the first time point to the second time point, and the opening/closing unit 170 is maintained in the closed state after the second time point is exceeded. Accordingly, the opening/closing unit 170 reduces the load applied to the shading motor 130 by suppressing the pressure increase in the flow path 40 over the period from the first time point to the second time point.

The opening/closing unit 170 in the present embodiment discharges the compressed air 4 in the flow path 40 from the opening 174 to the outside of the flow path 40 through the through hole 172 over the period from the first time point to the second time point. In addition, after the rotation speed exceeds the predetermined rotation speed, the opening 174 is closed by the closing portion 176 so that the opening/closing unit 170 stops the discharge of the compressed air 4 from the opening 174. Accordingly, even when the torque of the shading motor 130, which has been downsized, is reduced at the predetermined rotation speed or less, the load applied to the shading motor 130 is reduced by the opening/closing unit 170, and thus the shading motor 130 can be rotated in excess of the predetermined rotation speed (step S3).

Here, also during the period from the first time point to the second time point described above, a part of the compressed air 4 is discharged from the nozzle 110, and thereby the pressure increase in the flow path 40 is suppressed. However, “suppression of the pressure increase in the flow path caused by the discharge of a part of the compressed air from the nozzle” does not correspond to “suppression of the pressure increase in the flow path” in “the step of reducing the load applied to the shading motor by suppressing the pressure increase in the flow path over a period from the first time point at which the shading motor starts driving to the second time point at which the rotation speed of the rotor reaches the predetermined rotation speed” in the present disclosure.

That is, “suppression of the pressure increase in the flow path” in “the step of reducing the load applied to the shading motor by suppressing the pressure increase in the flow path over a period from the first time point at which the shading motor starts driving to the second time point at which the rotation speed of the rotor reaches the predetermined rotation speed” in the present disclosure means that the pressure increase in the flow path is suppressed separately from “suppression of the pressure increase in the flow path caused by the discharge of a part of the compressed air from the nozzle” described above.

As described above, the graph showing the relationship between the rotation speed and the torque of the shading motor in FIG. 7 is merely an example, and does not limit the characteristics of the shading motor. The characteristics of the shading motor, such as a torque or a maximum rotation speed, are arbitrarily set in accordance with the specifications of the motor or the frequency of a commercial power supply.

In the nebulizer 1 and the chemical atomization method according to the first embodiment of the present disclosure, the opening/closing unit 170 is provided as the load reduction mechanism 50 that suppresses the pressure increase of the compressed air 4 in the flow path 40 at the predetermined rotation speed or lower of the shading motor 130, whereby the load applied to the shading motor 130 due to the pressure increase of the compressed air 4 can be reduced at the predetermined rotation speed or less of the shading motor 130 which has been downsized. Accordingly, the load applied to the shading motor 130, which has been downsized, can be reduced, and thus the shading motor 130 can be rotated at the predetermined rotation speed or higher.

In the nebulizer 1 and the chemical atomization method according to the first embodiment of the present disclosure, the opening/closing unit 170 as the load reduction mechanism 50 allows the rotor 131 to rotate at the predetermined rotation speed or higher even when the shading motor 130 having a small size is used. Accordingly, even when the shading motor 130 having a small size is used, the chemical solution 2 can be sufficiently atomized.

In the nebulizer 1 and the chemical atomization method according to the first embodiment of the present disclosure, since the opening/closing unit 170 as the load reduction mechanism 50 is provided on the flow path 40, it is possible to reduce the load applied to the shading motor 130 until the rotor 131 of the shading motor 130 reaches the predetermined rotation speed. Thus, the use of the shading motor 130 having a small size can be enabled with a simple configuration in which the opening/closing unit 170 is provided on the flow path 40.

In the nebulizer 1 and the chemical atomization method according to the first embodiment of the present disclosure, since the opening/closing unit 170 as the load reduction mechanism 50 can be configured by a diaphragm valve having a simple configuration, it is possible to contribute to reduction of manufacturing cost and downsizing of the nebulizer 1.

In the nebulizer 1 and the chemical atomization method according to the first embodiment of the present disclosure, since the opening/closing unit 170 is a diaphragm valve, it is possible to easily adjust a pressure serving as a reference for the open state and the closed state of the opening/closing unit 170 by changing the thin film portion 173 constituting a part of the diaphragm valve.

### Second Embodiment

Hereinafter, a nebulizer and a chemical atomization method according to a second embodiment of the present disclosure will be described. Since the nebulizer and the chemical atomization method according to the second embodiment of the present disclosure are different from the nebulizer 1 and the chemical atomization method according to the first embodiment of the present disclosure in the configuration of the load reduction mechanism, description of the same configurations as those of the nebulizer 1 and the chemical atomization method according to the first embodiment of the present disclosure will not be repeated.

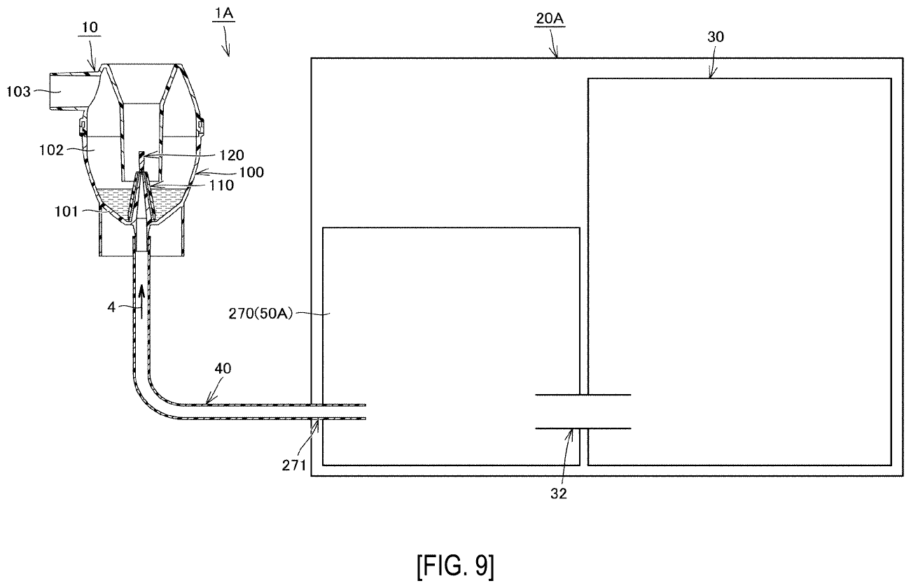

FIG. 9 is a schematic diagram illustrating a configuration of the nebulizer according to the second embodiment of the present disclosure. As illustrated in FIG. 9, a nebulizer 1A according to the second embodiment of the present disclosure includes the nebulizer kit 10, a main body portion 20A, the compressor 30, the flow path 40, and a load reduction mechanism 50A.

The load reduction mechanism 50A in the present embodiment is configured by a buffer tank 270 provided on the flow path 40.

The buffer tank 270 stores the compressed air 4 therein. The compressed air 4 compressed by the compressor 30 flows into the buffer tank 270 from the discharge port 32. The compressed air 4 flows from a flow port 271 to the nebulizer kit 10 via the flow path 40.

As compared with a case in which the buffer tank 270 is not provided on the flow path 40, the volume in the flow path 40 for accommodating the compressed air 4 is increased by providing the buffer tank 270, and thus a pressure increase due to the compressed air 4 in the flow path 40 is suppressed.

In the nebulizer 1A and the chemical atomization method according to the second embodiment of the present disclosure, since the buffer tank 270 is provided on the flow path 40 to increase the volume on the flow path 40 for accommodating the compressed air 4, the pressure increase in the flow path 40 can be suppressed. Accordingly, the load applied to the shading motor, which has been downsized, can be reduced, and the shading motor can be rotated at the predetermined rotation speed or higher.

In the nebulizer 1A and the chemical atomization method according to the second embodiment of the present disclosure, the buffer tank 270 as the load reduction mechanism 50A allows the rotor to rotate at the predetermined rotation speed or higher even when a shading motor having a small size is used. Accordingly, even when the shading motor having a small size is used, the chemical solution 2 can be sufficiently atomized.

In the nebulizer 1A and the chemical atomization method according to the second embodiment of the present disclosure, since the buffer tank 270 as the load reduction mechanism 50A is provided on the flow path 40, it is possible to reduce the load applied to the shading motor until the rotor of the shading motor reaches the predetermined rotation speed. Thus, the use of a small-sized shading motor can be enabled with a simple configuration in which the buffer tank 270 is provided on the flow path 40.

### Third Embodiment

Hereinafter, a nebulizer and a chemical atomization method according to a third embodiment of the present disclosure will be described. Since the nebulizer and the chemical atomization method according to the third embodiment of the present disclosure are different from the nebulizer 1 and the chemical atomization method according to the first embodiment of the present disclosure in the configuration of the load reduction mechanism, description of the same configurations as those of the nebulizer 1 and the chemical atomization method according to the first embodiment of the present disclosure will not be repeated.

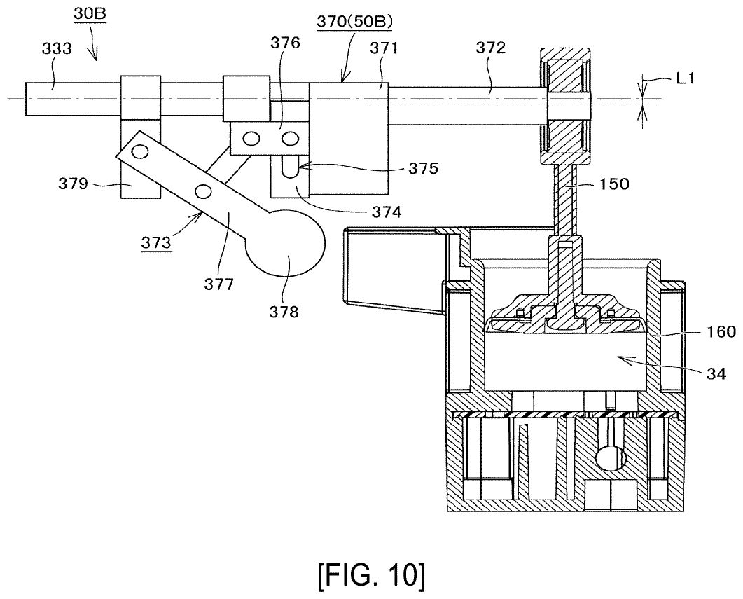

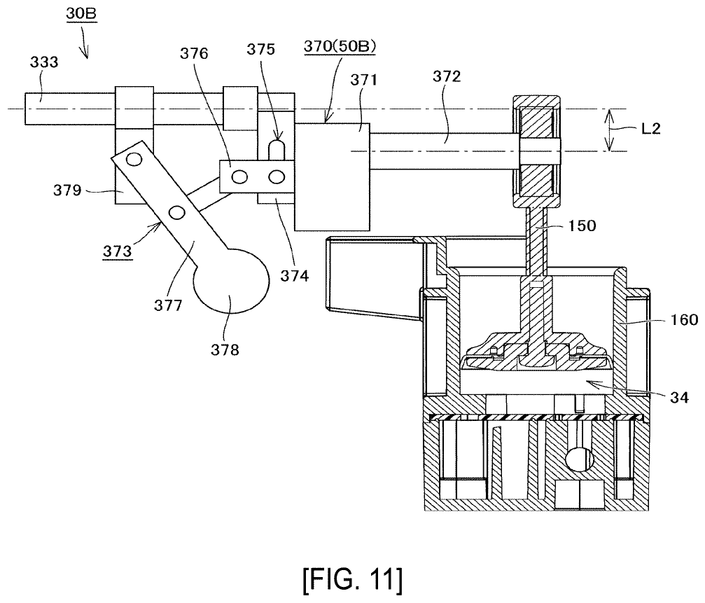

FIG. 10 is a schematic diagram illustrating a configuration when a crank in a compressor included in a nebulizer according to the third embodiment of the present disclosure rotates at a low speed. FIG. 11 is a schematic diagram illustrating a configuration when the crank in the compressor included in the nebulizer according to the third embodiment of the present disclosure rotates at a high speed.

As illustrated in FIG. 10 and FIG. 11, the nebulizer according to the third embodiment of the present disclosure includes a nebulizer kit, a main body portion, a compressor 30B, a flow path, and a load reduction mechanism 50B.

The compressor 30B includes a rotor shaft 333, a crank 370, and a link mechanism 373. The rotor shaft 333 is connected to a rotor (not illustrated).

The load reduction mechanism 50B according to the third embodiment of the present disclosure is configured by the crank 370. The crank 370 includes an eccentric shaft portion 371 and a connection shaft portion 372. The eccentric shaft portion 371 is connected to the connection shaft portion 372. The eccentric shaft portion 371 and the connection shaft portion 372 can be eccentric from the rotor shaft 333 by the link mechanism 373.

The link mechanism 373 includes a first member 374, a second member 376, and a third member 377.

The first member 374 is provided with an elongated hole 375. The second member 376 can move along the elongated hole 375 in a direction orthogonal to the rotation axis of the rotor shaft 333. The second member 376 is connected to the eccentric shaft portion 371. A weight 378 is provided at a tip of the third member 377. The third member 377 is connected to the rotor shaft 333 by a support member 379.

The compressor 30B in the present embodiment is configured to increase a compression ratio in the pump chamber 34. As the rotation speed of the rotor increases, the eccentricity of the crank 370 with respect to the rotor increases. Specifically, when the rotation speed of the rotor shaft 333 increases, the weight 378 of the third member 377 receives a centrifugal force in a direction orthogonal to the rotation axis of the rotor shaft 333. Accordingly, the second member 376 moves in the elongated hole 375 of the first member 374, whereby the eccentric shaft portion 371 and the connection shaft portion 372 move in the direction orthogonal to the rotation axis of rotor shaft 333. As a result, the eccentricities of the eccentric shaft portion 371 and the connection shaft portion 372 are changed.

As illustrated in FIG. 10 and FIG. 11, for eccentricities L1 and L2 of the crank 370, the eccentricity L2 during rotation at a high speed is larger than the eccentricity L1 during rotation at a low speed. The distance of the reciprocating motion of the piston 150 increases due to the increase in the eccentricity of the crank 370 with the increase in the rotation speed of the rotor, and thus the compression ratio of the compressed air 4 in the pump chamber 34 increases. That is, the compression ratio in the pump chamber 34 is lower during rotation of the crank 370 at a low speed than during rotation at a high speed, and thus the pressure increase in the flow path during rotation at a low speed is suppressed.

In the nebulizer and the chemical atomization method according to the third embodiment of the present disclosure, since the compressor 30B is provided with the crank 370 having a variable eccentricity, the compression ratio of the compressed air is increased with the increase in the rotation speed of the rotor, whereby the load applied to the shading motor due to the pressure increase of the compression air can be reduced at a predetermined rotation speed or less of the shading motor. Accordingly, the load applied to the shading motor, which has been downsized, can be reduced and the shading motor can be rotated at the predetermined rotation speed or higher. In the nebulizer and the chemical atomization method according to the third

embodiment of the present disclosure, the crank 370 as the load reduction mechanism 50B allows the rotor to rotate at the predetermined rotation speed or higher even when a small-sized shading motor is used. Accordingly, even when the small-sized shading motor is used, the chemical solution can be sufficiently atomized.

In the nebulizer and the chemical atomization method according to the third embodiment of the present disclosure, since the crank 370 having a variable eccentricity as the load reduction mechanism 50B is provided at the compressor 30B, it is possible to reduce the load applied to the shading motor until the rotor of the shading motor reaches the predetermined rotation speed. Thus, the use of a small-sized shading motor can be enabled with a simple configuration in which the crank 370 described above is provided at the compressor 30B.

The nebulizer according to each embodiment of the present disclosure has been described as a mobile and portable nebulizer, but the present disclosure can be applied not only to a mobile and portable nebulizer but also to a stationary nebulizer.

### Supplementary Notes

As described above, the present embodiments include the following disclosures.

### Configuration 1

A nebulizer including:
a compressor (30, 30B) including a shading motor (130) as a power source and being provided with a discharge port (32) that discharges compressed air (4) generated by driving the shading motor (130);
a nozzle (110) that injects the compressed air (4) generated by the compressor (30); an atomization unit (120) including a baffle (121) disposed so as to face the nozzle (110), the atomization unit (120) generating an aerosol (3) by adding a chemical solution (2) to the compressed air (4) injected from the nozzle (110) and spraying the compressed air (4) added with the chemical solution (2) onto the baffle (121); and a flow path (40) connecting the discharge port (32) and the nozzle (110), the shading motor (130) including a rotor (131) and a stator (132), the stator being made of a soft magnetic material and having an apparent volume of 78000 mm³ or less,
the nebulizer further including a load reduction mechanism (50) that reduces a load applied to the shading motor (130) by suppressing a pressure increase in the flow path (40) over a period from a first time point at which the shading motor (130) starts driving to a second time point at which a rotation speed of the rotor (131) reaches a predetermined rotation speed.

### Configuration 2

The nebulizer according to Configuration 1, wherein an opening/closing unit (170) is provided on the flow path (40), the opening/closing unit enabling discharge of the compressed air (4) in the flow path (40) to an outside of the flow path (40) in an open state and disabling discharge of the compressed air (4) in the flow path (40) to an outside of the flow path (40) in a closed state, and the opening/closing unit (170) is maintained in the open state over a period from the first time point to the second time point, and the opening/closing unit (170) is maintained in the closed state after the second time point is exceeded, so that the load reduction mechanism (50) is configured by the opening/closing unit (170).

### Configuration 3

The nebulizer according to Configuration 2, wherein the opening/closing unit (170) is formed of a diaphragm valve.

### Configuration 4

The nebulizer according to Configuration 1, wherein the load reduction mechanism (50A) is configured by a buffer tank (270) provided on the flow path (40).

### Configuration 5

The nebulizer according to Configuration 1, wherein the compressor (30B) further includes a piston (150), a cylinder (160), and a crank (370), the piston and the cylinder defining a pump chamber (34) in which the compressed air (4) is generated, the crank being connected to the rotor (131) and the piston (150) so as to convert a rotational motion of the rotor (131) into a reciprocating motion of the piston (150),
the compressor (30B) is configured such that an eccentricity of the crank (370) with respect to the rotor (131) increases as the rotation speed of the rotor (131) increases, and thereby a compression ratio in the pump chamber (34) increases, so that the load reduction mechanism (50B) is configured by the crank (370).

### Configuration 6

A chemical atomization method in a nebulizer for generating an aerosol (3) by injecting compressed air (4) from a nozzle (110) and spraying the compressed air onto a baffle (121) while adding a chemical solution (2) to the compressed air (4),
the nebulizer including:
a compressor (30) including a shading motor (130) as a power source and being provided with a discharge port (32) that discharges the compressed air (4) generated by driving the shading motor (130), and:
a flow path (40) connecting the discharge port (32) and the nozzle (110), the shading motor (130) including a rotor (131) and a stator (132), the stator being made of a soft magnetic material and having an apparent volume of 78000 mm³ or less,
the chemical atomization method including:
starting driving of the shading motor (130) (S1), and
reducing a load applied to the shading motor (130) by suppressing a pressure increase in the flow path (40) over a period from a first time point at which the shading motor (130) starts driving to a second time point at which a rotation speed of the rotor (131) reaches a predetermined rotation speed (S2).

The above-described embodiments disclosed herein are illustrative in all respects and do not constitute grounds for limited interpretation. Thus, the technical scope of the present disclosure shall not be interpreted only by the above-described embodiments. In addition, all modifications within the meaning and scope equivalent to the claims are included. In the description of the above-described embodiments, combinable configurations may be combined with each other.

### REFERENCE NUMERALS LIST

1, 1A Nebulizer; 2 Chemical solution; 3 Aerosol; 4 Compressed air; 10 Nebulizer kit; 20, 20A Main body portion; 21 Ventilation window; 30, 30B Compressor; 31 Suction port; 32 Discharge port; 33 Fan; 34 Pump chamber; 40 Flow path; 50, 50A, 50B Load reduction mechanism; 100 Case body; 101 Storage portion; 102 Aerosol discharge port; 103 Aerosol transport path; 104 Ventilation path; 110 Nozzle; 111 Hole; 120 Atomization unit; 121 Baffle; 122 Protrusion; 123 Solution suction portion; 130 Shading motor; 131 Rotor; 132 Stator; 133, 333 Rotor shaft; 134 Support base; 140, 370 Crank; 141 Eccentric portion; 150 Piston; 160 Cylinder; 161 Suction chamber; 162 Discharge chamber; 163 Valve; 170 Opening/closing unit; 171 Case; 172 Through hole; 173 Thin film portion; 174 Opening; 175 Sheet portion; 176 Closing portion; 177 Spring; 270 Buffer tank; 271 Flow port; 371 Eccentric shaft portion; 372 Connection shaft portion; 373 Link mechanism; 374 First member; 375 Elongated hole; 376 Second member; 377 Third member; 378 Weight; 379 Support member; H Height; T Thickness; W Width; L1, L2 Eccentricity.

What is claimed is:

1. A nebulizer comprising:
a compressor including a shading motor as a power source and being provided with a discharge port configured to discharge compressed air generated by driving the shading motor;
a nozzle configured to inject the compressed air generated by the compressor;
an atomization unit including a baffle disposed so as to face the nozzle, and being configured to generate an aerosol by adding a chemical solution to the compressed air injected from the nozzle and spraying the compressed air added with the chemical solution onto the baffle; and
a flow path connecting the discharge port and the nozzle, wherein
the shading motor includes a rotor and a stator, the stator being made of a soft magnetic material and having an apparent volume of 78000 mm³ or less, and
the nebulizer further includes a load reduction mechanism configured to reduce a load applied to the shading motor by suppressing a pressure increase in the flow path over a period from a first time point at which the shading motor starts driving to a second time point at which a rotation speed of the rotor reaches a predetermined rotation speed.

2. The nebulizer according to claim 1, wherein
an opening/closing unit is provided on the flow path, the opening/closing unit being configured to enable discharge of the compressed air in the flow path to an outside of the flow path in an open state and disable discharge of the compressed air in the flow path to the outside of the flow path in a closed state, and
the opening/closing unit is maintained in the open state over the period from the first time point to the second time point, and the opening/closing unit is maintained in the closed state after the second time point is exceeded, so that the load reduction mechanism is configured by the opening/closing unit.

3. The nebulizer according to claim 2, wherein the opening/closing unit is formed of a diaphragm valve.

4. The nebulizer according to claim 1, wherein the load reduction mechanism is configured by a buffer tank provided on the flow path.

5. The nebulizer according to claim 1, wherein
the compressor further includes a piston, a cylinder, and a crank, the piston and the cylinder defining a pump chamber in which the compressed air is generated, the crank being connected to the rotor and the piston so as to convert a rotational motion of the rotor into a reciprocating motion of the piston, and
the compressor is configured such that an eccentricity of the crank with respect to the rotor increases as the rotation speed of the rotor increases, and thereby a compression ratio in the pump chamber increases, so that the load reduction mechanism is configured by the crank.

6. A chemical atomization method in a nebulizer for generating an aerosol by injecting compressed air from a nozzle and spraying the compressed air onto a baffle while adding a chemical solution to the compressed air,
the nebulizer comprising:
a compressor including a shading motor as a power source and being provided with a discharge port configured to discharge the compressed air generated by driving the shading motor; and
a flow path connecting the discharge port and the nozzle,
the shading motor including a rotor and a stator, the stator being made of a soft magnetic material and having an apparent volume of 78000 mm³ or less, and
the chemical atomization method comprising:
starting driving of the shading motor; and
reducing a load applied to the shading motor by suppressing a pressure increase in the flow path over a period from a first time point at which the shading motor starts driving to a second time point at which a rotation speed of the rotor reaches a predetermined rotation speed.
